(19) United States
(12) Patent Application Publication
(10) Pub. No.: US 2009/0093499 A1
(43) Pub. Date: Apr. 9, 2009

(54) Pharmaceutical composition

(75) Inventors: Miha Tomaz Jaklic, Dol pri Ljubljani (SI); Klemen Naversnik, Vrhnika (SI)

Correspondence Address: SANDOZ INC, 506 CARNEFIE CENTER, PRINCETON, NJ, 08540, US

(73) Assignee: LEK PHARMACEUTICALS D.D., Ljubljana, SI (SI)

(21) Appl. No.: 12/158,413

(22) PCT Filed: Dec. 18, 2006
(86) PCT No.: PCT/EP2006/012180

§ 371 Date: Nov. 26, 2008

(30) Foreign Application Priority Data
Dec. 20, 2005 (SI) P200500344

(51) Int. Cl. A61K 31/505; A61P 3/06

(52) U.S. Cl. 514/256

(57) ABSTRACT

A chemically stable formulation of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof for oral use, such as tablets, capsules, powders, granules has been developed using the substances which stabilize against formation of degradation products: lactone and oxidation product.

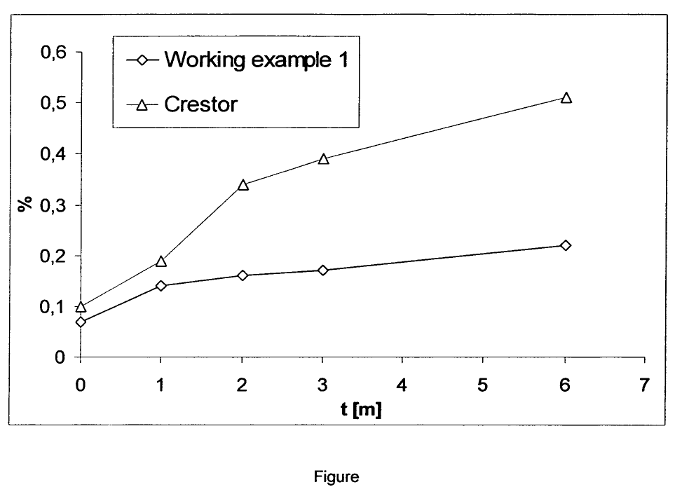

### FIELD OF THE INVENTION

Present invention from the field of pharmaceutics relates to pharmaceutical composition containing (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof (hereinafter “the agent”), especially the hemicalcium salt.

### BACKGROUND OF THE INVENTION

The agent is a 3-hydroxy-3-methylglutaryl (HMG) CoA reductase inhibitor known from EP 521471 and formulated into a pharmaceutical composition can be used for (manufacturing the medicament for) treating hypercholesterolemia, hyperlipidproteinemia and atherosclerosis. A major issue associated with the bulk agent or formulated into a composition is that it is particularly sensitive to degradation. The major degradation products formed (as known from U.S. Pat. No. 6,548,513) are the lactone (N-{4-(4-Fluoro-phenyl)-5-[2-(4-hydroxy-6-oxo-tetrtahydropyran-2-yl)-vinyl]-6-isopropyl-pyrimidin-2-yl}N-methyl-methanesulfonamide) and the oxidation product (7-[4-(4-fluorophenyl)-6-isopropyl-2-[methyl(methylsulfonyl)amino]pyrimidin-5-yl]-3-hydroxy-5-oxo-hept-6-enoic acid). Also, when exposed to light, the agent undergoes degradation to two diastereomeric cyclic products, described in U.S. 2005/0187234 A1. The mentioned degradations of the agent under conditions of humidity, acidity, oxygen and light is a challenge for the manufacture of a pharmaceutical formulation, stable enough for ordinary storage conditions.

This stabilization of the agent or chemically similar compounds, especially those belonging to HMG-CoA reductase inhibitors, could be achieved by controlling pH in a formulation (by addition of components such as a carbonate or bicarbonate) and by adding to composition a stabilizing inorganic salt, particularly tribasic tribasic calcium phosphate. Antioxidants, such as butylated hydroxytoluene may also be used to hinder oxidation of the agent. Another option is to stabilize a pharmaceutical composition using an amino sugar. Pharmaceutical composition of the agent currently marketed under name Crestor contains 5, 10, 20, or 40 mg of the agent, tribasic calcium phosphate as the stabilizing inorganic salt and the following inactive ingredients: microcrystalline cellulose, lactose monohydrate, crospovidone, magnesium stearate, hypromellose, triacetin, titanium dioxide, yellow ferric oxide, and red ferric oxide.

### DESCRIPTION OF THE FIGURE

FIGURE represents the comparison of the amount of the main degradation product (lactone form of the agent, as measured by HPLC), which is formed if the compositions corresponding to currently marketed formulation and the composition in accordance with our invention are subjected to accelerated stability program (as proposed by ICH guidelines: 40° C. and 75% relative humidity, stored in the primary package). The y axis represents the % of the formed degradation product (lactone), and x axis the time in months.

### DISCLOSURE OF THE INVENTION

In an aspect the invention provides a pharmaceutical composition comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof characterized in that it contains less than 0.05% as measured by HPLC of the 7-[4-(4-fluorophenyl)-6-isopropyl-2-[methyl(methylsulfonyl)amino]pyrimidin-5-yl]-3-hydroxy-5-oxo-hept-6-enoic acid.

In additional aspect the invention is a pharmaceutical composition comprising the agent, characterized in that it contains less than 0.5% as measured by HPLC of the N-{4-(4-Fluoro-phenyl)-5-[2-(4-hydroxy-6-oxo-tetrtahydropyran-2-yl)-vinyl]-6-isopropyl-pyrimidin-2-yl}N-methyl-methanesulfonamide and also less than 0.05% as measured by HPLC of the 7-[4-(4-fluorophenyl)-6-isopropyl-2-[methyl(methylsulfonyl)amino]pyrimidin-5-yl]-3-hydroxy-5-oxo-hept-6-enoic acid.

In a specific embodiment the invention is a pharmaceutical composition comprising the agent, characterized in that at least one of the ingredients is chosen from the first group consisting of: corn starch, silicified microcrystalline cellulose, croscarmellose sodium, and hypromellose and/or from the second group consisting of corn starch, mannitol, hydroxypropyl cellulose and hypromellose.

Specifically in an embodiment the pharmaceutical composition according will comprise silicified microcrystalline cellulose, agent, corn starch in weight ratio 10:3-4:1-2. More specifically it will additionally comprise up to 5% of at least one lubricant, which may be selected from group consisting of talc and glyceryl behanate

Yet more specifically the pharmaceutical composition will comprise the agent, silicified microcrystalline cellulose, corn starch, lactose, talc, colloidal silicon dioxide, glyceryl behanate and sodium stearyl fumarate in weight ratio 10:20-30:10-17:50-60:1-3:0-0.6:0-2:0-1.

The composition may film coated wherein said coating comprises HPMC, HPC, polyethylene glycol and talc.

In another aspect the invention represents a process for preparing a pharmaceutical composition comprising the agent with steps: a) mixing and screening of the agent and excipients, comprising silicified microcrystalline cellulose and corn starch to obtain homogeneous mixture; and b) (optional) granulation of powder mixture; and c) mixing of powder mixture (or granules) with lubricant; and d) compressing of powder mixture (or granules) into tablets; and e) (optional) coating of tablets prepared in preceding steps; in specific aspect wherein the weight ratio of agent: silicified microcrystalline cellulose: corn starch is 10:10 to 40:2 to 20.

Additionally the invention is a process for preparing a pharmaceutical composition comprising the hemicalcium salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid characterized in that the process comprises following steps: a) dry blending said hemicalcium salt and excipients mixture, wherein this mixture comprises lactose, silicified microcrystalline cellulose and corn starch; b) (optionally) mixing therein additional excipients; c) mixing therein a lubricant selected from sodium stearil fumarate or glyceryl behanate, specifically glyceryl behanate; d) compressing obtained powder mixture into tablets; e) (optionally) coating of tablets prepared in preceding steps, specifically in amounts said hemicalcium salt: 5-20% wt; lactose: 40-60% wt; silicified microcrystalline cellulose: 20-30% wt; and corn starch: 1 to 25% wt relative to the weight of the composition and (optionally) film coating.

Invention is embodied in the use of the pharmaceutical composition as described above for treating hypercholesterolemia, hyperlipidproteinemia and atherosclerosis and also in the use of the agent together with silicified microcrystalline cellulose and corn starch for manufacturing of a medicament for treating hypercholesterolemia, hyperlipidproteinemia and atherosclerosis.

In another aspect the inventions is the use of silicified microcrystalline cellulose and corn starch for stabilization of a pharmaceutical composition comprising hemicalcium salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid, specifically wherein said silicified microcrystalline cellulose and corn starch are together present in an amount 10-70% relative to the weight of pharmaceutical composition.

The invention is embodied in a novel pharmaceutical composition having the following advantages: the composition inhibits in long term (i.e. during the shelf life of a medicinal product) the formation of the agent's degradation products; the formulation is not alkaline in nature (measured by the pH of an aqueous dispersion of the formulation, which is 6,2) neither are any basifying agents added to hinder the degradation of the agent; the pharmaceutical composition allows a technically feasible process of creating a formulation with suitable biopharmaceutical properties.

Thus the lack of alkaline ingredients on one hand minimizes the potential of impaired in-vivo absorption of the agent due to the changes of the gastrointestinal pH and on the other side such composition is advantageous for patient because an alkaline composition would have adverse effects on gastric mucosa. The pH of the aqueous solution or dispersion of the composition in accordance with our invention will be substantially neutral, preferably between 6 and 8, as determined if a tablet containing 40 mg of agent is dispersed in 40 ml of water and measured by glass electrode pH meter. Compositions can be manufactured by established technological procedures, preferably direct compression or wet granulation followed by tabletting and film coating for manufacturing of finished dosage form (e.g. tablet) and at the same time demonstrates suitable biopharmaceutical properties such as comparable dissolution and/or bioequivalence to Crestor.

The present invention combines in a pharmaceutical composition the agent with the ingredients that stabilize the agent. Ingredients are selected according to two stabilizing properties. In the first group are the ingredients which were found to inhibit oxidation of the agent: corn starch, silicified microcrystalline cellulose, croscarmellose sodium, and hypromellose. In the second group are the ingredients which were found to inhibit the formation of the lactone form of the agent: corn starch, mannitol, hydroxypropyl cellulose and hypromellose. The formation of the degradation products of the agent under the influence of light can also be additionally hindered using pharmaceutically acceptable pigments or colorants, for instance in a tablet coating.

Preferably selection of ingredients from both groups and a protection from light results in a pharmaceutical composition wherein the agent is stabilized. In such formulation, the agent stays stable with respect to oxidation, formation of the lactone and formation of degradation products, preferably over a period of years, more preferably months.

In an embodiment the invention comprises a pharmaceutical composition comprising the agent, one or more ingredients from the first group (oxidation inhibiting ingredients), one or more ingredients from the second group (lactonization inhibiting ingredients) and one or more fillers (also known as diluents), binders, disintegrants or lubricants. Additionally, conventional excipients may be added: for example preservatives, silica flow conditioners, antiadherents and stabilizers. It will be appreciated that a particular excipient may act different roles in a pharmaceutical composition, e.g. as a filer, a binder and a disintegrant.

Typically the agent will be present in a weight amount within the range of 1 to 50%, preferably 3 to 30%. Typically the combined amount of stabilizing substance selected form the above first group and the above second group will be up to 90%, preferably 10 to 70%. The above stabilizing substances can also have a function of a filer (diluents), binder, or disintegrant. Typically one or more additional fillers may be present in amount up to 90% by weight, preferably 30 to 70%. Suitable additional fillers include, for example, lactose, cellulose and its derivatives (e.g. microcrystalline cellulose, powdered cellulose), modified starches, polyols, inorganic salts, or any other fillers commonly used in the art. Typically one or more binders will be present in an amount up to 90% by weight preferably 20 to 70%. Suitable binders include, for example, polyvinylpyrrolidone, gum acacia, gum tragacanth, guar gum, pectin, microcrystalline cellulose, methylcellulose, carboxymethylcellulose, hydroxypropyl methylcellulose, hydroxyethyl cellulose, hydroxypropyl cellulose, gelatin and sodium alginate. Suitable disintegrants include, for example, crosscarmellose sodium, crospovidone, sodium starch glycollate, hydroxypropyl methylcellulose and hydroxypropyl cellulose. Suitable lubricants include, for example, magnesium stearate, stearic acid, palmitic acid, calcium stearate, talc, carnauba wax, hydrogenated vegetable oils, mineral oil, glyceryl behanate, polyethylene glycols and sodium stearyl fumarate.

In a preferred embodiment a composition will contain from 4 to 11% of agent; from 10 to 50%, preferably in sum around 40% of stabilizing substances selected from the first group consisting of corn starch, silicified microcrystalline cellulose, croscarmellose sodium, and hypromellose and second group consisting of com starch, mannitol, hydroxypropyl cellulose and hypromellose. The stabilizing substances will be preferably silicified microcrystalline cellulose, corn starch and sodium starch glycolate, preferably in weight ratio 10:1-2:0-2. The composition may additionally comprise from 20 to 80%, preferably around 40 to 60% of lactose filler and up to 5% of lubricants, preferably talc, gylceryl behanate and sodium stearyl fumarate.

In a preferred embodiment the weight ratios of agent to silicified microcrystalline cellulose, corn starch, lactose, talc, colloidal silicon dioxide, glyceryl behanate and sodium stearyl fumarate will be 10:10-40:2-20:30-70:1-10:0-0.6:0-3:0-2. Most preferably the above ratios will be 10:20-30:10-17:50-60:1-3:0-0.6:0-2:0-1.

The pharmaceutical composition of the invention may be prepared using standard techniques and manufacturing processes generally known in the art, for example by dry blending the components. The components of the blend prior to blending, or the blend itself, may be passed through a mesh screen. Conveniently a lubricant, may also be added to the blend and blending continued until a homogeneous mixture is obtained. The mixture is then compressed into tablets. Alternatively, a wet granulation technique can be employed.

Pharmaceutical compositions comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid hemicalcium salt and ingredients from aforesaid first group and second group are prepared by dry blending the agent and all excipients (inactive ingredients) except lubricant in a double cone mixer. Next a lubricant, in one embodiment glyceryl behenate, and in another embodiment sodium stearyl fumarate is added to the mixture and blended for a short period of time, such as needed to substantially homogenize the mixture, e.g. up to 5 minutes. The mixture is then compressed into tablets and film coated with a conventional coating composition consisting of film forming polymer such as hypromellose or hydroxypropyl cellulose, film softener such as polyethylene glycol, pigments, talc.

A typical composition in accordance with our invention will comprise (wt.)

| | |
|---|---|
| agent (hemicalcium salt) | 5-20% |
| lactose | 40-60% |
| silicified microcrystalline cellulose | 20-30% |
| corn starch | 1-25% |

and further:

| | |
|---|---|
| a glidant (talc and/or colloidal silicium dioxide) | 0.5-5% |
| a lubricant (sodium stearyl fumarate and/or glyceryl behanate) | 0.1-3% |

and optionally further

| | |
|---|---|
| sodium starch gylcolate | 0-5% |

A tablet coating may then be applied, for example by spray-coating, with a water-ethanol based film coating formulation. Coating ingredient combinations are readily available. In an embodiment of the invention coatings containing pigments or colorants reduce the rate of formation of photo degradation products of the agent.

### Experimental Part

The bulk agent is subjected to stress conditions, such as elevated temperature (40° C. and above), elevated humidity (75% or higher relative humidity, open dish conditions), oxygen atmosphere or solutions with different pH. An HPLC analysis, capable of resolving the agent and its degradation products is then employed, to quantify the amount (given as a mass percentage relative to the agent) of degradation products in the stressed samples. Good chromatographic resolution can be achieved on a C18 reversed phase column with acidic phosphate buffer and an increasing gradient of acetonitrile and tetrahydrofuran. Degradation products are quantified using UV detection at 242 nm. The reporting limit of the degradation products has been set at 0.05%. The % reported for HPLC analysis are in general area %.

When bulk (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid hemicalcium salt is exposed to stress conditions the compound chemically degrades, showing its instability as demonstrated by following findings:

| | amount of | amount of |
|---|---|---|
| | lactone | oxidation |
| stress condition | (%) | product (%) |
| aqueous solution of the agent, buffered to pH = | 2.30 | <0.05 |
| 5 at room temperature (24 hours) | | |
| the agent under oxygen at 60° C. for 14 days | 0.17 | 0.71 |

The results of exposing to the same stress condition a mixture of agent and commonly used excipients such as lactose, crosslinked carboxymethylcellulose sodium or crosslinked PVP are substantially similar to results of the bulk agent subjected to the stress conditions.

However exposing mixtures of agent and the ingredients from the aforementioned first and second group, the amounts of lactone formed are substantially lower then as with the agent alone, showing their stabilizing property.

When a binary mixture (w 1:1) of the agent and any of the ingredients from the first group is stored under oxygen at 60° C. for 14 days, no oxidation product is formed compared to 0.71% formed on exposing the bulk agent.

Two working examples of stable formulations are presented, followed by two reference examples (first with commonly used tableting excipients and second including an alkalizing excipient).

### WORKING EXAMPLE 1

The following pharmaceutical composition is a novel composition, prepared by the process as described above, using ingredients from both two groups of stabilizing ingredients.

| Ingredient | Function | w % |
|---|---|---|
| The Agent (calcium salt) | active | 9.2 |
| Lactose | filler | 55.9 |
| Silicified Microcrystalline Cellulose | filler, binder, disintegrant, | 24.4 |
| | active stabilizer | |
| Corn Starch | filler, binder, disintegrant, | 3.6 |
| | active stabilizer | |
| Sodium Starch Glycolate | disintegrant, | 2.4 |
| | active stabilizer | |
| Talc | glidant | 2.7 |
| Glyceryl Behanate | lubricant | 1.8 |

The pH of the formulation is 6.2. The amount of lactone is only 0.50% after 14 days at 40° C. and 75% relative humidity (open dish). The oxidation product is not formed.

The amount of lactone does not increase above 0.25% after 6 months at 40° C. in impermeable package. The amount of the oxidation product after 6 months at 40° C. in impermeable package is only 0.05% or below. Comparatively the amounts of lactone and oxidation product in a pharmaceutical composition of the agent such as the one currently marketed (FIGURE) are 0.51% and 0.38%, respectively.

The tablet film coating (2.5% coating on weight of coated tablet) consisting of hypromellose, hydroxypropyl cellulose, polyethylene glycol, pigments and talc had no substantial effect on formation of lactone or oxidation product.

### WORKING EXAMPLE 2

| Ingredient | Function | w % |
|---|---|---|
| The Agent (calcium salt) | active | 9.2 |
| Lactose | filler | 48.9 |
| Colloidal Silicum Dioxide | glidant | 0.3 |
| Silicified Microcrystalline Cellulose | filler, binder, disintegrant, | 25.0 |
| | active stabilizer | |
| Corn Starch | filler, binder, disintegrant, | 15.0 |
| | active stabilizer | |
| Talc | glidant | 1.0 |
| Sodium Stearyl Fumarate | lubricant | 0.6 |

Tablets are film coated (2.5% coating on weight of coated tablet) consisting of hypromellose, hydroxypropyl cellulose, polyethylene glycol, pigments and talc.

The pH of the formulation is 6.3. The amount of lactone is only 0.35%-0.43% after 14 days at 40° C. and 75% relative humidity (open dish). The oxidation product is not formed.

### REFERENCE EXAMPLE 1

The following is a composition comprising agent and commonly used excipients as suggested by a lactose producer—DMV. Tablets are prepared according to same process as in above working examples. Amount of degradation products indicate instability of the agent in a composition.

| Ingredient | Function | w % |
|---|---|---|
| The Agent (calcium salt) | active | 9.4 |
| Lactose Anhydrous | filler, binder | 41.7 |
| Colloidal Silicium Dioxide | glidant | 0.3 |
| Lactose, Sieved | filler | 41.7 |
| Croscarmellose Sodium | disintegrant | 4.0 |
| Talc | glidant | 1.0 |
| Glyceryl Behanate | lubricant | 1.8 |

The amount of lactone is 2.32% after 14 days at 40° C. and 75% relative humidity (open dish).

### REFERENCE EXAMPLE 2

The following is a composition comprising agent and commonly used excipients as taught by a textbooks in this category: Pharmaceutical dosage forms: Tablets vol. 1 (Herbert, Lieberman, Lachman, and Schwartz; Marcel Dekker, New York and Basel; second edition, 1989. To this composition an alkalizing agent was added. Tablets are prepared according to same process as in above working examples.

| Ingredient | Function | w % |
|---|---|---|
| The Agent (calcium salt) | active | 12.8 |
| Sodium carbonate decahydrate | alkalizing agent | 1.8 |
| Lactose Anhydrous | filler, binder | 46.1 |
| Colloidal Silicium Dioxide | glidant | 3.7 |
| Microcrystalline Cellulose | filler, binder, disintegrant | 30.9 |
| Croslinked Povidone | disintegrant | 2.8 |
| Glyceryl Behanate | lubricant | 1.9 |

The composition shows the agent is stable in presence of alkalizing agent. The amount of lactone is 0.07% after 1 month at 40° C. and 75% relative humidity, open dish. The good stability of the sample is believed due to its alkalinity (pH=9,9)

### REFERENCE EXAMPLE 3

In parallel the stability of the currently marketed product has been assessed. The amount of lactone has increased from 0.10% to is 0.35% after 1 month at 40° C. and 75% relative humidity, and from 0.10% to is 0.40% after 10 days at 60° C. exposed to oxygen. The amount of oxidation product remain the same 0.3% under first conditions and increased to 0.4% under second.

1. A pharmaceutical composition comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof, containing less than 0.05% as measured by HPLC of the 7-[4-(4-fluorophenyl)-6-isopropyl-2-[methyl(methylsulfonyl)amino]pyrimidin-5-yl]-3-hydroxy-5-oxo-hept-6-enoic acid.

2. The pharmaceutical composition according to claim 1 containing less than 0.5% as measured by HPLC of the N-{4-(4-Fluoro-phenyl)-5-[2-(4-hydroxy-6-oxo-tetrtahydropyran-2-yl)-vinyl]-6-isopropyl-pyrimidin-2-yl)}-N-methyl-methanesulfonamide.

3. A pharmaceutical composition comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof, where at least one of the following additional ingredients is chosen from a group consisting of: corn starch, mannitol, hydroxypropyl cellulose, silicified microcrystalline cellulose, croscarmellose sodium, and hypromellose.

4. (canceled)

5. The pharmaceutical composition according to claim 1 comprising silicified microcrystalline cellulose and corn starch.

6. The pharmaceutical composition according to claim 1 comprising silicified microcrystalline cellulose, (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof, corn starch in weight ratio 10:3-4:1-2.

7. The pharmaceutical composition according to claim 6 comprising up to 5% of at least one lubricant.

8. The pharmaceutical composition according to claim 7 wherein the lubricant is selected from group consisting of talc and glyceryl behanate.

9. A pharmaceutical composition comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof, silicified microcrystalline cellulose, corn starch, lactose, talc, colloidal silicon dioxide, glyceryl behanate and sodium stearyl fumarate in weight ratio 10:20-30:10-17:50-60:1-3:0-0.6:0-2:0-1.

10. The pharmaceutical composition according to claim 1, wherein a pharmaceutically-acceptable salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid is the hemicalcium salt.

11. A pharmaceutical composition comprising a hemicalcium salt of (E)-7-[4-(4-fluorophenyl)-6-isopropyl-2-[methyl(methylsulfonyl)amino)pyrimidin-5-yl]-(3R, 5S)-3,5-dihydroxyhept-6-enoic acid; lactose; silicified microcrystalline cellulose, and corn starch.

12. The pharmaceutical composition according to claim 11, wherein said hemicalcium salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid is present in amount 5-20% wt; lactose in amount 40-60% wt; silicified microcrystalline cellulose in amount 20-30% wt; and corn starch in amount 1 to 25% and optionally sodium starch gylcolate in amount 0-5% wt.

13. The pharmaceutical composition according to claim 11, comprising in addition at least one glidant selected from talc or colloidal silicium dioxide.

14. The pharmaceutical composition according to the claim 13, wherein said glidant is present in total amount from 0.5 to 5% wt.

15. The pharmaceutical composition according to claim 11, comprising in addition at least one lubricant selected from sodium stearil fumarate or glyceryl behanate.

16. The pharmaceutical composition according to claim 15, wherein said lubricant is present in total amount from 0.1 to 3% wt.

17. The pharmaceutical composition according to claim 11, which is coated by a film coating.

18. The pharmaceutical composition according to claim 17, wherein said coating comprises HPMC, HPC, polyethylene glycol and talc.

19. The pharmaceutical composition according to claim 1, wherein pH of the aqueous solution or dispersion of the said composition will be substantially neutral.

20. The pharmaceutical composition according to claim 1, wherein pH of the aqueous solution or dispersion of the said composition will be between 6 and 8, as measured if a tablet containing 40 mg of agent is dispersed in 40 ml of water and measured by glass electrode pH meter.

21. A process for preparing a pharmaceutical composition comprising (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt where the process comprises the following steps:
a) mixing and screening of the (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt and excipients, comprising silicified microcrystalline cellulose and corn starch to obtain homogeneous mixture;
b) (optionally) granulating of powder mixture;
c) mixing of powder mixture or granules with lubricant;
d) compressing of powder mixture or granules into tablets;
e) (optionally) coating of tablets prepared in preceding steps.

22. The process according to claim 21, wherein the weight ratio of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt: silicified microcrystalline cellulose: corn starch is 10:10 to 40:2 to 20.

23. The process according to claim 21, wherein the lubricant in step c) is selected from glyceryl behenate or sodium stearil fumarate and is added to the mixture in step c) and blended.

24. The process according to claim 21, wherein a pharmaceutically-acceptable salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid is the hemicalcium salt.

25. A process for preparing a pharmaceutical composition comprising the hemicalcium salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid where the process comprises the following steps:
a) dry blending the hemicalcium salt of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid and excipients mixture, wherein this mixture comprises lactose, silicified microcrystalline cellulose and corn starch;
b) (optionally) mixing therein additional excipients;
c) mixing therein a lubricant selected from sodium stearil fumarate or glyceryl behanate;
d) compressing obtained powder mixture into tablets;
e) (optionally) coating of tablets prepared in preceding steps.

26. The process according to claim 25, wherein the amounts by weight to the weight of final compositions are:
hemicalcium salt of (E)-7-[4-(4-fluorophenyl)-6-isopropyl-2-[methyl (methylsulfonyl) amino]pyrimidin-5-yl]-(3R, 5S)-3,5-dihydroxyhept-6-enoic acid: 5-20%;
lactose: 40-60%;
silicified microcrystalline cellulose: 20-30%; and
corn starch: 1 to 25%.

27. The process according to claim 25, wherein the lubricant is glyceryl behanate.

28. A method of using silicified microcrystalline cellulose and corn starch for stabilization of a pharmaceutical composition comprising hemicalcium salt of (E)-7-[4-(4fluorophenyl)-6-isopropyl-2-[methyl(methylsulfonyl)amino]pyrimidin-5-yl]-(3R, 5S)-3,5-dihydroxyhept-6-enoic acid.

29. The use according to claim 28, wherein said silicified microcrystalline cellulose and corn starch are together present amount 10-70% relative to the weight of pharmaceutical composition.

30. The use according to claim 28, wherein pH of the aqueous solution or dispersion of the said composition will be between 6 and 8, as measured if a tablet containing 40 mg of agent is dispersed in 40 ml of water and measured by glass electrode pH meter.

31. A method of using the pharmaceutical composition according to claim 1 for treating hypercholesterolemia, hyperlipidproteinemia and atherosclerosis.

32. A method of using (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt together with silicified microcrystalline cellulose and corn starch for manufacturing of a medicament for treating hypercholesterolemia, hyperlipidproteinemia and atherosclerosis.

33. The use according to claim 32 where the weight ratios of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt: silicified microcrystalline cellulose: corn starch are 10:10-40:2-20.

34. The use according to claim 32, wherein pH of the aqueous solution or dispersion of the composition will be between 6 and 8, as measured if a tablet containing 40 mg of agent is dispersed in 40 ml of water and measured by glass electrode pH meter.

35. The pharmaceutical composition according to claim 1, wherein (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof stays stable with respect to oxidation, formation of the lactone and formation of degradation products over a period of a few months.

36. The pharmaceutical composition according to claim 3, containing (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof,
where the composition contains less than 0.5% as measured by HPLC of the N-{4-(4-Fluoro-phenyl)-5-[2-(4-hydroxy-6-oxo-tetrtahydropyran-2-yl)-vinyl]-6-isopropyl-pyrimidin-2-yl}-N-methyl-methanesulfonamide and less than 0.05% as measured by HPLC of the 7-[4-(4-fluorophenyl)-6-isopropyl-2-[methyl(methylsulfonyl)amino]pyrimidin-5-yl]-3-hydroxy-5-oxo-hept-6-enoic acid after subjecting to stability testing at 40° C. and 75% relative humidity for 6 months, stored in the primary package.

37. The pharmaceutical composition according to claim 36, wherein the % as measured by HPLC refer to percentage relative to amount of (E)-7-[4-(4-fluorophenyl)-6-isopropyl-2-[methyl(methylsulfonyl)amino]pyrimidin-5-yl]-(3R, 5S)-3,5-dihydroxyhept-6-enoic acid or a pharmaceutically-acceptable salt thereof.
